(19) United States
(12) Patent Application Publication
(10) Pub. No.: US 2022/0169908 A1
(43) Pub. Date: Jun. 2, 2022

(54) UREOLYSIS-INDUCED CALCIUM CARBONATE PRECIPITATION FOR SEALING CHANNELS AND OTHER USES

(71) Applicants: MONTANA STATE UNIVERSITY, Bozeman, MT (US); MONTANA EMERGENT TECHNOLOGIES, INC., Butte, MT (US)

(72) Inventors: Adrienne J. PHILLIPS, Bozeman, MT (US); Robin GERLACH, Bozeman, MT (US); Alfred B. CUNNINGHAM, Bozeman, MT (US); Lee H. SPANGLER, Bozeman, MT (US); Arda AKYEL, Bozeman, MT (US); Catherrin M. KIRKLAND, Bozeman, MT (US); Dwight Randall HIEBERT, Butte, MT (US)

(73) Assignees: MONTANA STATE UNIVERSITY, Bozeman, MT (US); MONTANA EMERGENT TECHNOLOGIES, INC., Butte, MT (US)

(21) Appl. No.: 17/441,336

(22) PCT Filed: Mar. 25, 2020
(86) PCT No.: PCT/US20/24596

§ 371 Date: Sep. 21, 2021

Related U.S. Application Data
(60) Provisional application No. 62/823,917, filed on Mar. 26, 2019.

(51) Int. Cl. C09K 8/42; C04B 28/10; C12Q 1/58

(52) U.S. Cl. CPC C09K 8/42; C12Q 1/58; C04B 28/10

(57) ABSTRACT

Methods of conducting ureolysis-induced calcium carbonate precipitation with a heat-treated cell preparation, methods for preparing the heat-treated cell preparation, and related materials. The methods of conducting ureolysis-induced calcium carbonate precipitation include precipitating calcium carbonate at a location by introducing urea, calcium, and a heat-treated cell preparation comprising active urease enzyme to the location. The urease enzyme hydrolyzes the urea to ammonium carbonate, and the calcium reacts with the carbonate to form a calcium carbonate precipitate at the location. The methods of preparing the heat-treated cell preparation include heating a urease-producing cell preparation at a temperature and for a time sufficient to inactivate at least a portion of the cells in the urease-producing cell preparation while maintaining at least some urease activity of urease made by the cells in the urease-producing cell preparation

### CROSS-REFERENCE TO RELATED APPLICATIONS

Priority is hereby claimed to provisional application Ser. No. 62/823,917, filed Mar. 26, 2019, which is incorporated herein by reference.

### STATEMENT REGARDING FEDERALLY SPONSORED RESEARCH

This invention was made with government support under Grant Nos. DE-SC0010099 and DE-FE0026513 awarded by the Department of Energy. The United States Government has certain rights in the invention.

### FIELD OF THE INVENTION

The invention is directed to ureolysis-induced calcium carbonate precipitation, particularly with the use of a heat-treated cell preparation, as well as materials and additional methods associated therewith.

### BACKGROUND

Ureolysis-induced calcium carbonate (CaCO₃) precipitation (UICP) has been proposed for use in a range of engineering applications. Examples of such applications include amending or improving construction materials (De Muynck, et al. 2010), cementing porous media (Whiffin et al. 2007, van Paassen and Ghose et al. 2010), and environmental remediation (Fujita et al. 2010, Lauchnor et al. 2013, Phillips and Gerlach et al. 2013). UICP has also been proposed for use in oil and gas applications, such as improving wellbore and caprock integrity and other functions (Dupraz and Parmentier et al. 2009, Cuthbert et al. 2013, Phillips et al. 2013, Phillips and Gerlach et al. 2013, Phillips and Lauchnor 2013, Phillips et al. 2015, Phillips et al. 2016, Cunningham et al. 2009, Cunningham et al. 2011, Cunningham et al. 2014, Cunningham et al. 2015, U.S. Pat. No. 9,739,129). An advantage of the in situ mineral precipitation strategies in oil and gas applications is the use of low viscosity fluids that promote mineral precipitation to “grow a seal” in situ in the near wellbore environment instead of injecting higher viscosity fluids such as cements or gels. UICP has the potential to be utilized in place of traditional cement or grout in the subsurface for remediating wellbore integrity, sealing fractures in concrete and rock formations utilized for fluid storage (e.g. CO2, natural gas, or H2), controlling flow paths for oil and gas recovery, or creating subsurface barriers for water pollution control (Cuthbert et al. 2013, Fujita et al. 2008, Mitchell et al. 2010, Lauchnor et al. 2013).

UICP involves the ureolysis (degradation) of urea to form ammonium carbonate, which then reacts with calcium to form calcium carbonate (Eq. 1).

CO(NH₂)₂+2H₂O+Ca²⁺↔2NH₄⁺+CaCO₃(s) (1)

The calcium carbonate can form crystalline structures that adhere to surrounding materials (e.g., sand, rock, cement), thereby modifying the strength, porosity, and permeability of the material receiving treatment.

UICP can take the form of enzyme-induced calcium carbonate precipitation (EICP), microbial-induced calcium carbonate precipitation (MICP), thermally induced calcium carbonate precipitation (TICP), and chemically induced calcium carbonate precipitation (CICP). In EICP, ureolysis is mediated by purified or semi-purified (e.g., crude plant meal, such as ground jack bean meal, soy bean meal, chick pea meal) urease enzyme, which catalyzes the hydrolysis of urea into ammonium and carbonate (U.S. Pat. No. 6,401,819 to Harris et al., U.S. Pat. No. 9,150,775 to Östvold, U.S. Pat. No. 10,215,007 to Bansal, EP 1980604 to Lundgaard). In MICP, ureolysis is mediated by urease enzyme actively produced by live urease-producing microbes (U.S. Pat. No. 5,143,155 to Ferris et al. U.S. Pat. No. 8,420,362 to Crawford et al., U.S. Pat. No. 8,460,458 to Jonkers, U.S. Pat. No. 9,683,162 to Ravnas, U.S. Pat. No. 9,739,129 to Cunningham et al., U.S. Pat. No. 9,809,738 to Luke et al., U.S. Pat. No. 10,138,406 to Ravnas, US 20060216811A1 to Cunningham et al., US 20110011303 to Jonkers, WO 2008120979 to van Paassen, WO 2010075503A1 to Cunningham et al.). In TICP, ureolysis occurs through thermal decomposition of urea (U.S. Pat. No. 8,522,872 to Bour et al.). In CICP, ureolysis is mediated by catalysis with non-enzymatic catalysts.

MICP, mostly using the ureolytic bacterium Sporosarcina pasteurii, has been extensively researched (Stocks-Fischer et al. 1999, Whiffin et al. 2007, Phillips et al. 2016, Cunningham et al. 2014, Mitchell et al. 2006, DeJong et al. 2006, Tobler et al. 2012, van Paassen and Ghose et al. 2010). MICP has been shown to be effective at reducing permeability and sealing leakage fractures, including fractures in shale and sandstone (Cunningham et al. 2015, Phillips et al. 2016), and has been successfully implemented in large-scale field applications (Cuthbert et al. 2013, van Paassen and Ghose et al. 2010, Gomez et al. 2015). Demonstrations reported by Phillips et al. (Phillips et al. 2016, Phillips et al. 2018) showed the potential for MICP to seal subsurface leakage pathways at depths >300 m. In those studies, the subsurface fluid temperature was around 20° C., which is within the range for mesophilic microbial growth.

There are a number of potential problems with MICP, however. Temperature generally increases with increasing depth in the terrestrial subsurface. Many oil and gas applications such as permeability modification for improving recovery from unconventional formations will most certainly have to be performed at higher temperature conditions. To extend the application range, temperature-tolerant strategies will therefore need to be developed. Furthermore, increased pressures and harsher chemical environments, such as CO₂-saturated brines, may further limit the suitability of microbes. Finally, the introduction of live organisms into the environment might not always be permitted, as regulations may limit the amendment of subsurface environments with living microbes.

One strategy for overcoming these and other issues with MICP include using purified or semi-purified ureases obtained from plant-based or microbial-based sources. However, urease from plant-based sources is expensive and enzyme purification is cumbersome and costly.

Strategies for overcoming the aforementioned challenges are needed.

### SUMMARY OF THE INVENTION

The invention is directed, at least in part, to methods of conducting EICP using a heat-treated cell preparation.

One aspect of the invention is directed to methods of precipitating calcium carbonate at a location. The methods comprise introducing urea, calcium, and a heat-treated cell preparation comprising active urease enzyme to the location. The unease enzyme hydrolyzes the urea to ammonium carbonate, and the calcium reacts with the carbonate to form a calcium carbonate precipitate at the location.

In some versions, the heat-treated cell preparation includes, in addition to the active urease enzyme, heat-treated cells in the form of intact cells, non-intact remnants thereof, or a mixture thereof.

In some versions, the heat-treated cell preparation is produced by heating a urease-producing cell preparation.

In some versions, the heating comprises heating the urease-producing cell preparation at a temperature from about 50° C. to about 70° C. for a time from about 1 minute to about 30 minutes. In some versions, the urease-producing cell preparation comprises a urease-producing microbe. In some versions, the urease-producing microbe comprises Sporosarcina pasteurii. In some versions, the heating is sufficient to inactivate at least a portion of the cells in the preparation while maintaining at least some of the activity of the urease made by the cells. In some versions, the heating inactivates at least about 95% of the urease-producing cells in the urease-producing cell preparation. In some versions, the heating increases urease activity in the heat-treated cell preparation with respect to urease activity in the urease-producing cell preparation. In some versions, the heat-treated cell preparation is introduced to the location after the heating without purification or isolation of components therefrom.

In some versions, at least one of the urea and the calcium is introduced to the location in a fluid preparation separate from the heat-treated cell preparation.

In some versions, the location comprises a channel, and the urea, the calcium, and the heat-treated cell preparation is introduced into the channel in amounts and for a time sufficient to at least partially seal the channel. In some versions, the channel is a subterranean channel. In some versions, the channel is in fluid communication with a wellbore. In some versions, the channel comprises a channel in a subterranean formation. In some versions, the channel comprises a channel between a cement sheath and a well casing of a subterranean wellbore. In some versions, the channel comprises a channel between a cement sheath surrounding a well casing of a subterranean wellbore and a subterranean formation through which the subterranean wellbore is drilled. In some versions, the channel comprises a crack in a cement sheath surrounding a well casing of a subterranean wellbore.

Another aspect of the invention is directed to methods of preparing a heat-treated cell preparation. The methods comprise heating a urease-producing cell preparation at a temperature and for a time sufficient to inactivate at least a portion of the cells in the urease-producing cell preparation while maintaining at least some urease activity of urease made by the cells in the urease-producing cell preparation.

The objects and advantages of the invention will appear more fully from the following detailed description of the preferred embodiment of the invention made in conjunction with the accompanying drawings.

### BRIEF DESCRIPTION OF THE DRAWINGS

FIG. 1. EICP with microbially-derived isolated urease at 70° C. Suspensions of urease enzyme isolated from S. pasteurii microbes and urea/calcium-containing solutions were alternately injected into 1-mm gap between a cement-steel interface at 70° C. Permeability was reduced with the increasing number of pulses.

FIG. 2. Laboratory test of EICP with heat-treated cells. A sandstone/cement composite core soaked with CO₂ saturated brine conditions in a high-pressure system raised to 55° C. and 1200 psi was mineralized using heat-treated S. pasteurii cells (cells exposed to 60° C. for 11.8 minutes) followed by injections of urea/calcium solutions.

FIG. 3. Field test of EICP with heat-treated cells. Suspensions of heat-treated S. pasteurii cells (cells exposed to 60° C. for 8-13 minutes) and urea/calcium-containing solutions were alternately injected into an acid-treated well. Permeability expressed as flow to pressure ratio (gallons per minute/pounds per square inch (gpm/psi)) is shown.

FIG. 4. Ultrasonic imaging logs before and after EICP treatment of the well of FIG. 3. Black=primarily solids and some gas, white=liquid. Fluids were able to exit the wellbore in three potential locations indicated by the arrows (at 990, 1004, 1017-1019 feet below ground surface) due to previously drilled sidewall-cores and pre-existing perforations. Before production of CO₂ and EICP treatment, a channel was observed in the solid material detected behind the casing (hatched circle on left panel). After EICP treatment a significant amount of solid was detected (solid circles on right panel).

### DETAILED DESCRIPTION OF THE INVENTION

The present invention is directed in part to ureolysis-induced calcium carbonate precipitation (UICP). The UICP of the invention can be used for wellbore and fracture sealing, in addition to other uses.

Some aspects of the present disclosure are directed to methods of precipitating calcium carbonate at a location. The methods can comprise introducing urea, calcium, and a heat-treated cell preparation comprising active urease enzyme to a location. Calcium carbonate can precipitate at the location by virtue of the urea, calcium, and heat-treated cell preparation mixing at the location, the urease enzyme hydrolyzing the urea into ammonium carbonate, and the calcium reacting with the carbonate to form calcium carbonate at the location.

The heat-treated cell preparation can comprise a fluid (e.g., liquid, gel, etc.) preparation that includes, in addition to the active urease enzyme, heat-treated cells. The heat-treated cells can be in the form of intact cells, non-intact remnants thereof, or a mixture thereof. The heat-treated cells (intact cells, non-intact remnants, or mixture thereof) can be suspended in the fluid or attached (adhered) to a surface within the fluid.

The heat-treated cell preparation can be made by heating a urease-producing cell preparation. The urease-producing cell preparation can comprise urease-producing cells in a fluid. The urease-producing cells can be suspended in the fluid or attached to a surface within the fluid. Heating the urease-producing cell preparation can be carried out at any of a number of temperatures. Exemplary temperatures include temperatures of at least about 30° C., at least about 35° C., at least about 40° C., at least about 45° C., at least about 50° C., or at least about 55° C. to about 65° C., to about 70° C., to about 75° C., to about 80° C. or more, such as about 60° C. Heating the urease-producing cell preparation can be carried out for a time of at least about 1 minute, at least about 3 minutes, at least about 5 minutes, or at least about 8 minutes to about 20 minutes, to about 30 minutes, to about 45 minutes, to about 60 minutes, or more.

After the heating, the heat-treated cell preparation can be cooled from the heating temperature prior to introducing the preparation to the location. The heat-treated cell preparation can be cooled to a temperature less than about 65° C., less than about 60° C., less than about 55° C., less than about 50° C., less than about 45° C., less than about 40° C., less than about 35° C., less than about 30° C., less than about 25° C., less than about 20° C., less than about 15° C., less than about 10 ° C., less than about 5° C., or less than about 0° C. In the cooling, the heat-treated cell preparation may be frozen to temperatures as low as −80° C. or lower. However, in some versions, the heat-treated cell preparation is cooled to a temperature no lower than about −80° C., no lower than about −50° C., no lower than about −10° C., no lower than about 0° C., or no lower than about 1° C. After generating the heat-treated cell preparation, it is preferred, but not required, to avoid freezing the heat-treated cell preparation or at least to limit the number of freeze-thaw cycles.

The heat-treated cell preparation is preferably used in a “crude” form, e.g., without any or at least substantial purification or removal of any components therefrom. The term “crude” encompasses preparations to which additional material is added.

The urease-producing cells in the urease-producing cell preparation can comprise any cell, whether prokaryotic or eukaryotic, that naturally produces urease, does not naturally produce urease but is genetically modified to produce urease, or naturally produces urease and is genetically modified to enhance production of urease. The urease-producing cells in some versions can comprise urease-producing microbes. The urease-producing microbes can comprise urease-producing bacteria. The urease-producing bacteria can comprise Sporosarcina pasteurii.

The urease-producing cell preparation can itself be generated using the methods outlined in Phillips et al. 2016, which is incorporated herein by reference.

The heating can be sufficient to inactivate at least a portion of the cells in the preparation while maintaining at least some of the activity of the urease made by the cells. “Inactivate” used herein with reference to a cell means rendering the cell unable to grow or be cultured in or on a medium such as an agar plate or a liquid growth medium.

The urease-producing cell preparation can accordingly be heated to a temperature and for a time sufficient to inactivate at least a portion of the urease-producing cells. The heat-treated cells in the heat-treated cell preparation can comprise heat-inactivated cells. The heat-inactivated cells can be in the form of intact, inactivated cells, non-intact remnants thereof, or a mixture thereof. The heating in some versions can inactivate at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or about 100% of the cells in the urease-producing cell preparation. The heat-treated cell preparation can contain a number of live cells less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1° A, or about 0% of the number of live cells in the urease-producing cell preparation prior to the heating. The number of live cells can be quantitated by any of a number of methods known in the art, including counting cell colonies after plating on a petri dish.

The heating can maintain at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or about 100% of the urease activity present in the urease-producing cell preparation.

The heating in some versions of the invention can actually increase the urease activity in the heat-treated cell preparation with respect to the urease activity in the urease-producing cell preparation. It is hypothesized that this may occur by releasing active urease previously contained inside the cells into the surrounding fluid. Accordingly, the heat-treated cell preparation can have an increase in urease activity that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, or at least about 200% higher than the urease activity present in the urease-producing cell preparation. The increase in urease activity in the heat-treated cell preparation can be up to about 2-fold, up to about 5-fold, up to about 10-fold or more higher than the urease activity in the urease-producing cell preparation. Urease activity can be quantitated using any of a number of methods known in the art, such as with the use of the Urease Activity Assay Kit (Sigma Aldrich, St. Louis, Mo., Product Number MAK120).

The urea and calcium can be introduced to the location in the heat-treated cell preparation or in one or more fluid preparations separate from the heat-treated cell preparation. To prevent premature calcium carbonate precipitation, however, it is preferred that at least one of the urea and calcium be introduced to the location in one or more fluid preparations separate from the heat-treated cell preparation. A number of suitable formats are acceptable in this regard. In one format, the urea can be introduced in the heat-treated cell preparation and the calcium can be introduced in a separate fluid preparation. In another format, the calcium can be introduced in the heat-treated cell preparation and the urea can be introduced in a separate fluid preparation. In another format, the urea and the calcium can be introduced in a single fluid preparation separate from the heat-treated cell preparation. In another format, the urea and calcium can be introduced in separate fluid preparations that are each separate from the heat-treated cell preparation.

In some versions of the invention, the location can comprise a channel. The channel can be a channel through soil, rock, cement, or any other gap within or between one or more solid substrates. The urea, calcium, and heat-treated cell preparation can be introduced into the channel in amounts and for a time sufficient to at least partially seal the channel.

In some versions, the channel can be a subterranean channel. The subterranean channel can be a fracture in a formation, a crack within cement, a channel formed by debonding of cement from casing, or other gaps that may occur underground, whether in a natural formation or a human-made structure such as a well. The channel can be in fluid communication with a wellbore. “Fluid communication” as used herein refers to a mutual configuration between two elements such that fluid residing in a first element (such as a wellbore) can flow (by force of pressure, gravity, or any other force) to and into the second element (such as a subterranean channel) without interruption. “Wellbore” refers to the a hole that is drilled into the ground or any other solid surface. The channel can comprise a channel in a subterranean formation. The channel can comprise a channel between a cement sheath and a well casing of a subterranean wellbore. The channel comprises a channel between a cement sheath surrounding a well casing of a subterranean wellbore and a subterranean formation through which the subterranean wellbore is drilled. The channel can comprise a crack in a cement sheath surrounding a well casing of a subterranean wellbore. For a discussion of such types of channels, see U.S. Pat. No. 8,522,872 to Bour et al., which is incorporated herein by reference.

In some versions, the methods can be used in any application in which UICP is or can be employed. These include sealing hydraulically fractured rock, consolidating proppant placed in fractured rock, sealing leaks in wellbore encasements, or other applications. See, e.g., Phillips et al. 2016 and U.S. Pat. No. 9,739,129, among the other references cited herein. The methods outlined in these references, including methods of introducing fluid preparations (e.g., suspensions/solutions) to particular locations, preparation of live cell suspensions, preparation of urea and/or calcium solutions, and preparation of other elements, can be employed for use in the present invention. The heat-treated cell preparation of the present invention, for example, can be used in place of the cell suspensions/solutions or the enzyme suspensions/solutions in the methods described in the references cited herein.

Unconventional oil and gas recovery has succeeded in part because of the ability to increase permeability in tight source rock via fracturing. While this has led to large increases in resource recovery, the ability to modify fracture apertures to allow more accurate control of permeability in these rocks has the potential to dramatically increase recovery efficiency. The ability to control permeability along fractured shale flow paths, both in the presence and in absence of proppant, creates new opportunities for enhancing oil and gas recovery from unconventional reservoirs. Mineral precipitation has the potential to impact subsurface permeability by: (1) sealing fractures in thief zones to improve injected fluid sweeps, (2) stabilizing injected proppant to reduce proppant flow-back, increasing or maintaining the aperture under production pressure levels to increase stimulation volume or (3) sealing existing fracture apertures followed by re-fracturing to open formation access to previously unrecoverable oil and gas. The methods provided herein are tools to modify permeability to access and improve the recovery of gas and oil from unconventional formations.

As used herein, “biomineralization” refers generally to methods that employ enzymatic hydrolysis of urea, such as EICP and MICP.

“Fluid” is used herein to refer to any non-gaseous fluid, including liquids, gels, or other fluid media.

“Fluid preparation” is used herein to refer to any preparation comprising a fluid base, including solutions, suspensions, etc.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

The present examples show the use of UICP with heat-treated cells and other improvements. The mineral sealing technology can be used to mitigate wellbore or cap rock leakage and/or to seal fractured formations. The examples show the capacity of UICP to be employed under a variety of pressure, temperature, and chemical conditions.

### Example 1

### EICP with Microbial Derived Urease

Advanced mineralization sealing methods are aimed at developing low-viscosity, fluid-based mineral precipitation sealing technologies that address a wide range of subsurface temperature and depth conditions. Proof-of-principle data show that direct injection of the urease enzyme to promote calcium carbonate precipitation is applicable for temperatures up to at least 80° C. (140° F.).

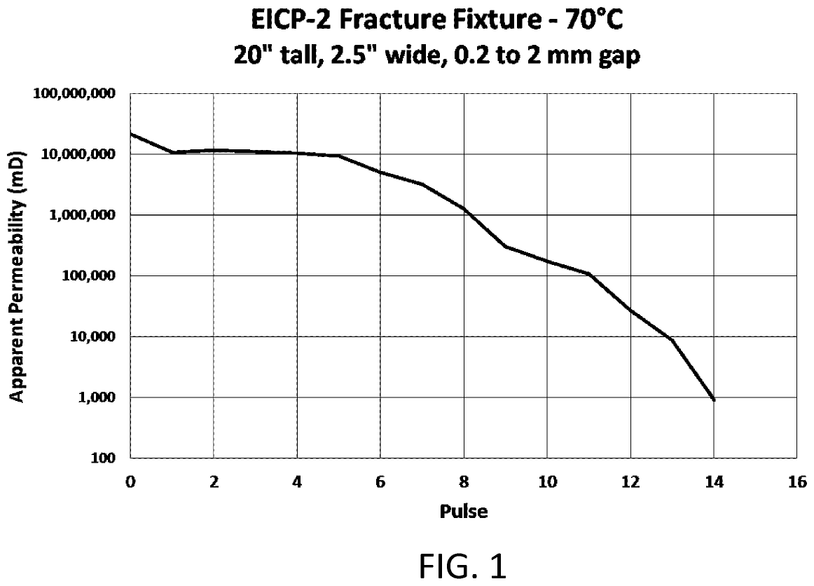

Batch experiments with plant- and microbially-based urease enzyme (Jack Bean Meal (JBM)) and S. pasteurii) were conducted at temperatures between 20 and 80° C. The experiments demonstrated optimum hydrolysis of 20 g/L urea at 60° C. within 60 minutes, with slower urea hydrolysis rates at lower temperatures. At higher temperatures, JBM urease showed increasing rates of thermal inactivation of the urease enzyme, resulting in incomplete urea hydrolysis. In the experimental system shown in FIG. 1, a 1-mm gap between a cement-steel interface was sealed using EICP with microbially-derived urease at 70° C. Briefly, urease enzyme isolated from S. pasteurii microbes was injected as a suspension into the aperture. Injection of the enzyme suspensions were alternated with urea/calcium containing solutions that promoted precipitation. Over the course of several days the permeability was reduced by more than four orders of magnitude (FIG. 1).

EICP with the S. pasteurii-derived urease was also conducted in an engineered fractured core system at 55° C. Significant permeabilty reduction (five orders of magnitude) was observed during the mineralization treatment over the course of 23 calcium injections. A large cap of mineral material was observed to form on the core. A significant difference was observed in the amount of open pore space in the engineered gap as detected by X-ray computed tomography (X-Ray CT).

### Example 2

### Heat-Treating Urease-Producing Cells

We have determined that urease-producing microbes can be heat-treated in a manner that inactivates them (where they are unable to continue to grow) without destroying urease enzyme activity. Heat-treating cells can permit enzyme-induced calcium carbonate precipitation (EICP) without injecting growing/live cells into the subsurface.

S. pasteurii cell suspensions were exposed to 60° C. for 11.8, 21.1, or 26.1 min. It was observed that the cells exposed to as little as 11.8 minutes of heating were unable to regrow while no significant reduction in the urease acitvity was observed.

### Example 3

### Laboratory Testing of EICP with Heat-Treated Cells in the Presence of CO₂

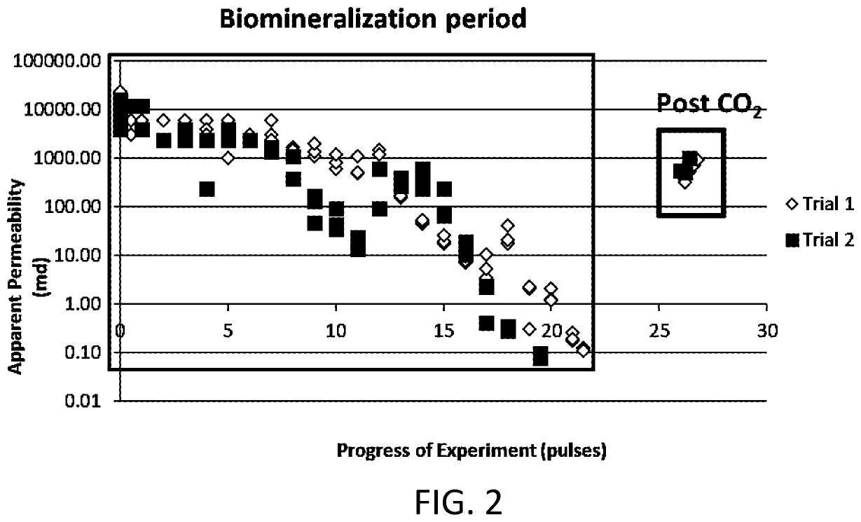

EICP using heat-treated cells (exposed to 60° C. for 11.8 minutes) was tested in a sandstone/cement composite core soaked with CO₂ saturated brine conditions in a high-pressure system raised to 55° C. and 1200 psi. After CO₂-affected brine flooding, the core was mineralized using heat-treated cells followed by injections of urea/calcium solutions. Post-mineralization, the core was again exposed to CO₂-affected brine to assess the strength of the mineral seal after exposure. Differential pressure and flow rate data were monitored and recorded over the course of the experiment. The apparent permeability of the core decreased 5-6 orders of magnitude over the course of the experiment (FIG. 2). Permeability increased after the subsequent CO₂ exposure but did not return to the original pre-mineralization permeability, indicating that the influence of the CO₂ exposure was limited. Final permeability values observed for both cores were comparable, indicating reproducible behavior.

### Example 4

Field Testing of EICP with Heat-Treated Cells in the Presence of CO₂

Field work was performed to test the use of heat-treated cells in EICP to seal a channel in wellbore cement in the presence of CO₂-impacted brine. The Gorgas well in Alabama was identified as a suitable well for the field testing. The Gorgas well was originally drilled to perform pilot-scale injections of CO₂. There are several locations in the well where leakage pathways exist in the wellbore cement. There are also several access points (sidewall core holes) in the wellbore to access those leakage pathways.

A rig was set up over the Gorgas well, and a mobile laboratory was used to process samples, grow microbes, heat-treat the cells, and mix calcium-urea solution.

The testing included the following steps: Sample the wellbore fluids, pump water and acid to create a channel in the wellbore cement; add acid and bicarbonate to generate CO₂ in the channel; and subsequently seal the channel with EICP mineralization.

During the field work, five key activities occurred:
1) Sampling of downhole fluids 4 times: immediately after placing tubing string, after addition of HCl and sodium bicarbonate to generate acidic conditions and CO₂, mid EICP, and at the end of the EICP treatment;
2) Heat-treating of cells (which can render cells inactive but preserves the activity of the urease enzyme) in the mobile laboratory, which was conducted with a heat-treating system using stainless steel coils and heated water;
3) Injection of the heat-treated cells and calcium-urea solution to promote mineralization in the wellbore channel;
4) Monitoring of flow-pressure relationships to determine success of sealing; and
5) Logging of the well before injection of HCl and sodium bicarbonate (CO₂ production) and after EICP treatment to assess the presence of solids in the channel.

Sampling Downhole Wellbore Fluids. Downhole fluid samples were collected using a sampling bailer. First, a field blank was collected, where distilled water was poured through bailer and collected in a dedicated disinfected sample-collection bucket in order to obtain a geochemical baseline and microbial community associated with the bailer and bucket. Samples of the downhole fluids were then collected for microbial community and geochemical analyses. Geochemistry samples were immediately analyzed for pH, temperature, conductivity, and dissolved oxygen. A portion of the sample was filtered and acidified, while another portion was only filtered before being stored on dry ice. Samples were collected: 1) prior to the experiments, 2) after the addition of HCl and sodium bicarbonate, 3) mid-EICP treatment, and 4) post-EICP treatment.

Results of the basic geochemical analysis performed in the field with a HACH multimeter are presented in Table 1. The “post CO₂ flowback” and “end EICP flowback” samples were collected by releasing pressure on the well which should have served to cause fluids behind the casing in the cement channel to flow back into the well. Static samples were collected in the wellbore without flowback. A low pH was measured in the “post CO₂ flowback” samples, pH 1.1-1.3, compared to the initial downhole samples that measured above 8. The dissolved oxygen in the samples was also variable ranging from 0.6-5.7 mg/L. This may not be entirely representative of the downhole conditions as the samples were exposed to air when they were released from the bailer and collected in the bucket.

TABLE 1
Geochemical analysis of sampled wellbore fluids.

| | | | Temp. | | EC | DO |
|---|---|---|---|---|---|---|
| Date | Time | Sample ID | (° F.) | pH | (mS/cm) | (mg/L) |
| Dec. 6, | 12:44 | bailer control | | 4.35 | 0.1335 | |
| 2018 | | | | | | |
| | 16:45 | downhole initial 1 | 60 | 8.4 | 23.7 | |
| | 17:00 | downhole initial 2 | 60 | 8.33 | | |
| Dec. 7, | 7:49 | downhole initial 3 | 60 | 8.41 | 14.46 | |
| 2018 | | | | | | |
| Dec. 10, | 9:30 | post CO₂ flowback | 55 | 1.35 | 28.3 | 4.33 |
| 2018 | | | | | | |
| | 9:50 | post CO₂ flowback | 56 | 1.11 | 44.6 | 4.31 |
| | 10:12 | post CO₂ flowback | 56 | 1.11 | 40.5 | 4.39 |
| Dec. 12, | 10:15 | mid-EICP static | 56 | 5.62 | 61 | 1.77 |
| 2018 | | | | | | |
| Dec. 13, | 12:15 | end EICP static | 63 | 6.47 | 48.9 | 0.62 |
| 2018 | | | | | | |
| | 12:30 | end EICP flowback | 66 | 7.21 | 8.96 | 5.7 |

Heal-Treating of Cells. Sporosarcina pasteurii cultures were grown to an average concentration of 1.7×10⁸ cfu/mL in the mobile laboratory in 15-gallon reactors equipped with aeration, ventilation, recirculation and temperature control. After approximately 24 hours of growth in the reactors, the cultures were combined and transferred to a holding tank from which they were pumped into the heat-treating system. Cells were heat-treated using stainless steel coiled tubing suspended in 60° C. water prior to cooling the heat-treated cell preparation by flowing the cells through tubing in a water tank cooled to below 30° C. to protect the urease enzyme from any inactivation The residence time for cells flowing through the stainless-steel coil in the heated water was 8-13 minutes, which inactivated 99.99% of the cells (determined using viable plate counts on agar plates) without inactivating the urease enzyme.

Heat-treated cell preparations were compared to actively growing cells and the heat-treated cells exhibited up to 58% higher ureolytic activity (according to conductivity measurements) than the growing/live cells. One explanation for this could be a partial breakdown and thus increased permeability of the bacterial cell walls allowing urea to enter the heat-treated cells more easily or allowing urease to be released from the cells, thus facilitating contact between the enzyme and urea. In the case of the live cells, transport of urea into the cells (or urease out of the cells) might be limited, thus becoming a rate-limiting step in urea hydrolysis.

Injection of Fluid Preparations. Urea and calcium solutions were mixed in a 30-gallon tank and pumped into the bailer, and heat-treated cells were transferred to a 15-gallon tank from the heat-treating coil system located in the mobile laboratory. Fluids were pumped into the bailer with transfer pumps and dedicated hoses. The bailer was filled with ˜2.8 gallons of the mineralization-promoting fluid preparations prior to delivery downhole. Bailers were delivered and returned to the surface in approximately 20-minute trip times. Approximately 62 gallons of heat-treated cells (22 bailers) and 84 gallons of calcium-urea solution (30 bailers) were injected over 4 days.

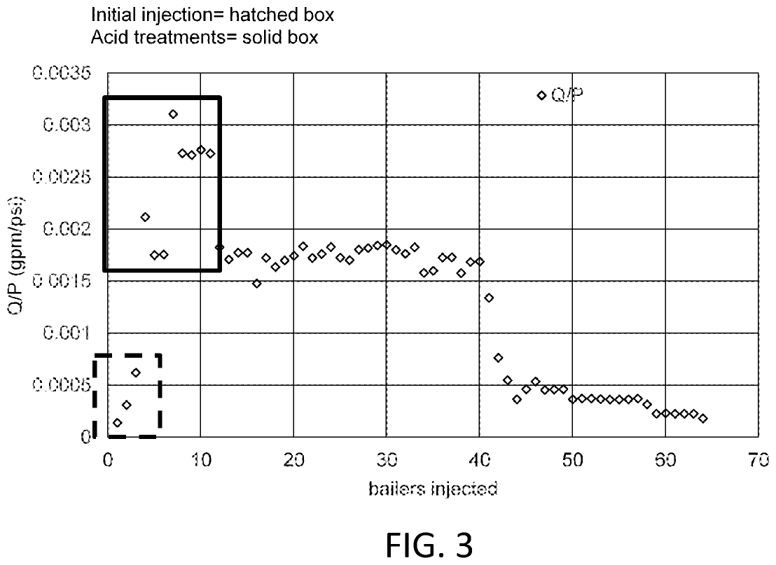

Monitoring Flow-Pressure Relationships. Pressure and flow rate were monitored and recorded as water was injected to push fluids that were delivered in the bailer (either acid, sodium bicarbonate, or EICP promoting fluid preparations) into the wellbore channel. The EICP treatment resulted in a 94% reduction of apparent permeability in the channel. Before EICP treatment but after acid and sodium bicarbonate injection, the channel conveyed 2.2 gpm at 708 psi. After EICP treatment, the final flow-pressure conditions were 0.2 gpm at 1104 psi. The flow to pressure ratio was plotted against the number of bailer injections and decreased from a maximum of 0.031 (gpm/psi) to 0.00018 (gpm/psi) as EICP treatment occurred (FIG. 3).

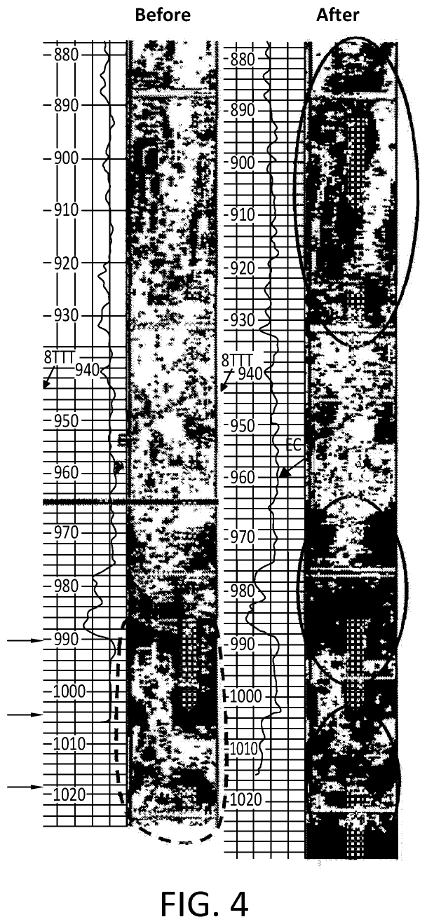

Logging the Wellbore. The well was logged twice. The well was first logged after acid treatment of the channel (pre-EICP treatment) and then logged again at the end of the experiment (post-EICP treatment). In the logging, it was observed that the acid treatment might have opened a channel that appeared to trend downward outside the casing. After EICP treatment, there was a noticeable increase in the percentage of solids in the channel in the region of the side wall cores (990-1019′ bgs) and up to 100+ feet above the injection point (FIG. 4).

The results of this experiment suggest that heat-treated microbes can be successfully employed in EICP in the presence of CO₂-impacted brine. The EICP with heat-treated microbes can seal leakage pathways, ensure storage of CO₂ in geologic carbon sequestration scenarios, seal channels between wellbore cement and steel interfaces, and/or seal any other underground channel for any other purpose.

### Example 5

### Additional Field Demonstrations of EICP with Heat-Treated Urease-Producing Cells

Additional field demonstrations of EICP with heat-treated urease-producing cells can be achieved with the following methods.

Field Site. A well can be chosen based on having su□cient permeability to inject fluids. A porosity of approximately 10% and a permeability of approximately 3 mD is suitable. The well can be cased completely to the bottom, and the casing in the target region can be perforated in order to access the formation. Perforation of the casing and the formation can occur with shots at 60-degree phasing within the zone from 340.7 to 341.1 m bgs. The perforations can have a ˜0.89 cm entry hole and extend ˜50.8 cm into the formation. After perforation and sump amendment (see below for details), the packer can be engaged to isolate the formation. The tubing string can be equipped with downhole pressure memory gauges (Schlumberger, USA) below the packer. A collar stop can be placed between two 1.2-m perforated pipes on which the bailer could land and open (see, e.g., FIG. 1 of Phillips et al. 2016). The tubing can be set into the well with the end of the perforated tubing at about 341.7 m. Water can be trucked from a plant to two holding tanks where it can be amended with NaCl (Mix-N-Fine, Minnesota, USA) to 2.4% final NaCl concentration (hereafter referred to as the brine). The flow rate from the Cat Model 310 (Cat Pumps, Minneapolis, Minn.) injection pump powered by a 5 HP 230 V motor with a variable speed chive can be monitored by a Ho□er flow meter (Ho□er Inc., North Carolina, USA) with an Omega (Omega Engineering Inc., Connecticut, USA) pressure data logger to record surface pressure. The injection pump can be connected to the tubing string to be able to pump brine into the subsurface.

To create injectivity into the formation, the formation can be stimulated by increasing the brine injection flow rate until a downhole pressure of 105.9 atm, when the formation's fracture initiation pressure is reached. An injection test can be performed by pumping brine at 1.9 L/min for 6 h, which results in a steady downhole pressure of 68 atm. The target flow rate of 1.9 L/min can be chosen for the EICP study to remain below fracture initiation pressure during the EICP treatment period. After the 6 h injection test, the well can be shut in for an 88-h pressure fallo□ test. From the pressure fallo□ data, it can be estimated that a horizontal radial fracture is created.

Density Amendment of Sump. Tubing (2⅞″ OD) can be strung down-hole with the packer unengaged. The bottom portion of the well (sump) can be located between perforations and the bottom plug which resides approximately 10 feet below. The sump can be filled sodium chloride-river water solution of 13% NaCl(Mix-N-Fine, Cargill Salt Division, Minnesota, USA) by pumping brine through the tubing string. The purpose of filling the sump with fluid of higher density than the other injection fluids is to encourage flow into the formation instead of injected fluids sinking into the bottom approximately 10 feet of the injection zone due to density differences.

Heat-Treated Cell Preparation. Frozen stocks of Sporosarcina pasteurii culture can be obtained and stored onsite on dry ice. To start a culture, filtered (0.45 μm bottle top filter Fisher Scientific, NJ, USA) BHI+Urea medium (37 g and 20 g/L respectively, culture starter medium) can be filled to 150 ml in 250-ml pre-sterilized plastic screw top flasks (VWR, PA, USA) and inoculated with 1 ml of a thawed frozen stock. The 150-ml cultures can be grown overnight and then transferred to 5-gallon collapsible carboys (Cole Parmer) filled to 4 gallons with the growth medium. The carboy growth medium can be prepared by dissolving 3 g/L Nutrient Broth (Research Products International), 10 g/L NH₄Cl (Amersco), 20 g/L Urea fertilizer (Par 4, Bridgewell Resources, OR, USA) and 24 g/L NaCl (Morton, Ill., USA) in distilled water in the collapsible carboy prior to inoculation. A stir bar can be added to the carboy and the entire carboy can be placed in a heated (20 degrees C.) Rubbermaid tub where it is stirred and the culture allowed to grow for approximately 24 hours. Continuous overnight cultures of S. pasteurii can be maintained throughout the experiment. Periodic samples can be drop plated on BHI+Urea agar plates to assess the microbial viability, microbial concentration, and the potential for a contamination to overtake the S. pasteurii culture. Cultures can be heat treated as described elsewhere herein. The live cell concentration in the heat-treated cell preparation can be assessed using colony forming unit (cfu) counts on brain heart infusion (BHI)+urea agar plates via the drop plate method (Herigstad et al. 2001) (37 g/L BHI, Becton Dickinson, New Jersey, USA), 20 g/L urea (Fisher Scientific, New Jersey, USA), 15 g/L Difco agar (Becton Dickinson, New Jersey, USA).

Pulsed Injection Strategy. All the EICP supporting substrates can be pre-weighed into quart and gallon sized plastic zip closure bags at the appropriate mass to reach the desired substrate concentrations after pumping brine following bailer delivery. Those bags of substrates were mixed with brine immediately prior to bailer delivery downhole as per the following injection schedule (listed as: bailer contents, pump run time (min), flow rate (L/min)): Sample #1, N/A, N/A; Heat-treated cell preparation #1, 20, 1.89; Calcium #1, 20, 1.89; Calcium #2, 20, 1.89; Calcium #3, 20, 1.89; Heat-treated cell preparation #2, 20, 1.89; Sample #2, N/A, N/A; Calcium #4, 10, 1.89; Calcium #5, 10, 1.89; Calcium #6, 14, 1.89; Calcium #7, 14, 1.89; Calcium #8, 20, 1.89; Heat-treated cell preparation #3, 20, 1.70; Calcium #9, 14, 1.89; Calcium #10, 14, 1.89; Calcium #11, 14, 1.70; Calcium #12, 14, 1.51; Calcium #13, 10, 1.89; Calcium #14, 20, 1.89; Heat-treated cell preparation #4, 18, 1.89; Calcium #15, 14, 1.89; Calcium #16, 14, 1.89; Calcium #17, 14, 1.89; Calcium #18, 14, 1.89; Calcium #19, 12, 1.89; Calcium #20, 14, 1.89; Calcium #21, 20, 1.89; Heat-treated cell preparation #5, 11,1.89; Brine, 5, 0.47; Sample #3, N/A, N/A; Calcium #22, 23.3, 0.53; Calcium #23, 13.5, 0.53; Heat-treated cell preparation #6, 14.45, 0.53; Calcium #24, 13.49, 0.53.

Field Test Design. The EICP field test can occur over 4 days using a pulsed injection strategy similar to the one described in Ebigbo 2012 with delivery of concentrated solutions via a slickline dump bailer. The bailer is a hollow tube with a valve on the bottom, which can be filled with di□erent fluid mixtures and then sent downhole where it sits down on the collar stop causing a pin to shear and the bottom bailer valve to open. Two types of fluid mixtures can be delivered with the bailer (1) heat-treated cell preparation prepared as described below amended with 24 g/L urea fertilizer (Potash Corporation, Illinois, USA) and (2) calcium-containing solution: 99 g/L CaCl₂ (OxyChem Ice Melt, Michigan, USA), 23.3 g/L NH₄Cl (BASF, New Jersey, USA) and 56 g/L urea mixed with brine. The concentration of the calcium-containing solution can be chosen such that the dilution with brine would yield substrate concentrations in the fracture supportive of EICP, (the targeted concentrations were: 24 g/L urea, 10 g/L NH₄Cl, and 39 g/L calcium chloride). The actual concentration achieved in the fracture after dilution can be based on the amount of brine pumped, which can average 28.7±7 L after each bailer delivery. The heat-treated cell preparation and calcium-containing solution were injected separately with a brine rinse between injections in order to minimize direct contact between the heat-treated cell preparation and calcium-containing solution potentially resulting in undesirable EICP within the 7.3 cm diameter tubing or the wellbore mixing zone.

The use of a 9.1 m long 5.1 cm diameter, 11.4 L, slickline dump bailer can be used an economical, conventional oil and gas field methodology to deliver the substrates to the subsurface. Substrates or microbial suspensions (heat-treated cell preparation) can be mixed in a tank at the ground surface and the bailer can be filled by pumping the solutions into the top of the bailer. The filled bailer can be lowered into the well into the region of the fracture where it opens to release the heat-treated cell preparation. The heat-treated cell preparation can be delivered into the fracture and formation by pumping brine through the tubing string. The calcium-containing solutions can then be delivered, each followed by brine dilution. Prior to an overnight shut in period, a second microbial heat-treated cell preparation can be delivered. Pressure and flow rate during injection and pressure fallo□ in between flowing periods can be monitored and recorded by the surface and subsurface pressure gauges. The bailer can be rinsed with brine between calcium containing solutions and microbial suspensions and at the end of each day. Each day, the well can be shut in overnight. Injections can occur for 4 days.

Sampling and Chemical Analysis. Prior to the initial injection, fluids in the well casing at 340 to 341 m bgs can be sampled using the Wireline Kuster Sampling tool (Schlumberger, Fla., USA). The Kuster sampler can be set on a timer to open after placement into the wellbore mixing zone and closed after 20 min. Once closed, the tool can be retrieved and the wellbore liquid sample can be collected in an autoclaved bottle (Nalgene, Thermo Fisher Scientific New York, USA). Additional samples can be collected after the initial injection. Portions of the sample can be used to assess the microbial community, concentration of urea and calcium, as well as pH. Concentration of urea can be assessed by a modified method of the Jung Assay (Phillips 2013, Jung et al. 1975). The pH can be assessed with a two-point calibrated meter (Accumet AP71, Fisher Scientific, New Jersey, USA). Calcium concentrations can be assessed using ion chromatography (IC) as previously described (Phillips and Lauchnor et al. 2013, Ebigbo et al. 2012). The formation water can also be chemically analyzed. Unfiltered samples from each mixing zone sample can be plated onto BHI+urea agar to assess culturability of microorganisms. Unique colonies that grow on the BHI+urea agar plates can be streaked for isolation on fresh agar plates, after which they can be inoculated into autoclaved BHI+urea liquid medium and cultured at room temperature. Identification of the isolates via 16S rRNA gene sequencing can then be performed (Phillips et al. 2016). A portion of the sample (350 mL) can be filtered through a 0.2 μm bottle top filter (Thermo Scientific, New York, USA).

Experiment Termination and Post-experiment Analysis. The experiment can be terminated when fluids can no longer be injected through the tubing without exceeding the threshold pressure (TP). The TP can be set at 81.6 atm to remain well below the initially observed fracture extension pressure (96.6 atm). Over the course of the experiment, the flow rate can be reduced from the original flow rate to prevent the pressure from increasing above the fracturing pressure during the treatment period. The pressure decay can be monitored by recording the decrease in well pressure for 5 min after shut in (pumping stopped). A 100% reduction would be equivalent to the well head pressure decreasing to atmospheric pressure indicating, for example, leakage or unobstructed flow into a formation. At the end of the experiment, a step rate test can be performed where pumping pressures are increased until the formation refractured.

After the EICP sealing, samples can be retrieved from the region of the fracture by drilling side wall cores below ground surface. A cement plug and piece of the casing can be retrieved, and the cement portion can be imaged using Xray Micro Computed Tomography (Micro-CT) (Sky Scan 1173, Bruker USA, Wisconsin, 100 kV, no filter). The cement core can also be analyzed with a Leica M205FA stereomicroscope using reflected white light and fluorescence, (DAPI cube, ex 350/50, em 460/50, Leica Microsystems, Illinois). Drilling mud type material can also be retrieved, which can be dried in a sterile Petri dish on the benchtop in the laboratory prior to being analyzed using X-ray powder di□raction spectrometry (XRD) (Scintag X-GEN 4000 XRD).

### Example 6

### Enzyme Kinetic & Inactivation Experiments

Inactivation temperatures of urease enzymes can be determined as follows.

Batch experiments can be carried out in digital shaking water baths operating at the desired experimental temperature (e.g., 20-80° C. or greater) at 70 rpm. The initial heating period to reach each temperature can be determined by measuring the temperature over time with an Omega CDS107 temperature probe. Time to reach 95% of the target temperature is preferably determined to be less than 3.5 minutes for each treatment. Experiments can be carried out in 30 mL glass bottles containing urease to which 10 mL of 40 g*L⁻¹ pre-heated urea solution can be added once the experimental temperature is reached. This creates a urease-urea mixture with final concentrations of 2.5 g*L⁻¹ urease and 20 g*L⁻¹ urea.

Batch experiments can be run in triplicate for durations of two to eight hours, depending on temperature, while measuring conductivity with an Omega CDS107 conductivity probe. In addition, two 60 μL samples can be taken for urea concentration analysis (as presented in Phillips 2013, modified from Jung et al. 1975) every 15 minutes up to two hours and then every 30 minutes up to eight hours. Less than 10% of the total experimental volume can be removed for sampling. As a positive control, treatments can also be run at 30° C. to monitor potential variations between different enzyme preparations. A urease-free control with 20 g*L⁻¹ urea can be run to assess abiotic hydrolysis within the experimental time. Abiotic hydrolysis of urea should be monitored to ensure it does not occur at the tested temperatures.

The modified Jung assay for urea and conductivity-based measurements can be used to monitor the rate of urea hydrolysis. The Jung assay can be performed in 96 well plates with absorbance measured at 505 nm to assess urea concentrations (Phillips 2013, Jung et al. 1975). Data can be correlated to conductivity readings taken in parallel at each temperature. The conductivity method can be used to measure the proportional increase in conductivity due to the conversion of non-ionic urea into ionic ammonium and (bi)carbonate ions during urea hydrolysis (Whiffin et al. 2007). The equation of the resulting correlation line of the combined triplicates can be used to convert the conductivity measurements to urea hydrolyzed (g*L⁻¹).

Enzyme inactivation can be determined by exposing 10 mL of 5 g*L⁻¹ urease suspensions to temperatures between 50 and 80° C. for 0.5 to 168 hours. Exposed urease suspensions can be cooled down rapidly on ice at pre-determined times and stored at 4° C. until utilized in batch experiments to determine the remaining enzyme activity (A). To determine A, each 10 mL sample of thermally-exposed urease suspension can be warmed to 30° C. and mixed with 10 mL of a 40 g*L⁻¹ urea solution at 30° C.; A can be estimated by determining the average urea hydrolysis rate based on the difference in the initial and residual urea concentrations after 2 hours (Equation 2). Here, U₀ and U_{ΔT} are the urea concentrations initially and after two hours (120 minutes), respectively, and ΔT is the time of the kinetic experiment (2 hours).

$$A = \frac{[ {U}_{0} - {U}_{Δ t} ]}{Δ t} \qquad Equation 2$$

Urea hydrolysis promoted by microbial enzyme sources has been shown to follow a first order rate expression as suggested by Ferris et al. (2003) and for plant-based sources as summarized by Handley-Sidhu et al. (2013) for urea concentrations near 20 g*L⁻¹ (Equation 3). Initial comparisons can be based on the first order rate coefficients (kᵤᵣₑₐ) determined from 120-minute batch studies for each temperature, calculated using the hydrolysis rates from experimental data (Equation 3).

$$\frac{d U}{1 t} = - {k}_{urea} [ U ] \qquad Equation 3$$

Where dU is the differential change in urea concentration, dt is the differential change in time and kᵤᵣₑₐ is the apparent first order reaction rate coefficient (min⁻¹).

Ureases can become inactivated at elevated temperatures such as those above 50° C. Hence, the model can be modified to include both changes in urea concentration (U) and enzyme activity (A) (Equation 4) where kᵤᵣₑₐ can be temperature-dependent and A temperature- and time-dependent.

$$\frac{d u}{d t} = - {k}_{urea} [ U ] [ A ] \qquad Equation 4$$

Here, the reaction equation is second order overall, first-order with respect to urea concentration (U) and first-order with respect to enzyme activity (A). The inactivation of the urease enzyme at elevated temperatures can be modeled using the activity term (A), a function of temperature and time. To determine the activity term, especially at the elevated temperature, three inactivation models of differing complexity can be considered, one single-step and two multi-step inactivation models. These evaluated models are graphically shown in Table 2.

TABLE 2
Graphical representation of inactivation models and their
corresponding theoretical inactivation pathway(s).

| Model | Mechanism |
|---|---|
| First order | 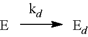 |
| Series-parallel | 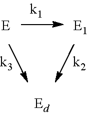 |
| Series-type | 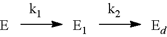 |

Some literature suggests that enzyme inactivation kinetics can be described using a first-order inactivation model (Equation 5), which describes a one-step irreversible inactivation of the enzyme from its native form to an inactivated form (Aymard et al. 2000, Henley et al. 1984, Illeová et al. 2003, Anthon et al. 2002). In some cases, a first-order model may describe an enzyme's inactivation mathematically but may not be exact from a mechanistic standpoint. In these cases, higher order or more complex inactivation models, including series-parallel and series type models, might better describe the pathway (Sadana et al. 1988). In a series-parallel model (Equation 6), the native enzyme follows one of two paths towards the inactivated form, (1) a two-step series path that assumes a partially inactivated isozyme (E₁) during inactivation and (2) a single step path toward complete inactivation (E_{d}). The series-type inactivation model (Equation 7) follows a two-step inactivation pathway through a partially inactivated isozyme (E₁) to a completely inactivated form (E_{d}). In each of these higher order models, kinetic coefficients k1, k2, and k3 are the reaction rate coefficients of enzyme inactivation from the native form (E) to the isozyme form (E₁) to the inactive form (E_{d}) (see Table 2 for a graphical representation). k1 is the kinetic rate constant for isomerization and k2 and k3 are the kinetic rate constants for complete inactivation. An additional parameter included in the biphasic models is a β term which represents an activity ratio of E and E₁. All of the k values described above and noted in the equations below are dependent on temperature.

$$\begin{matrix} A = {A}_{o} {e}^{- {k}_{d} * t} & Equation 5 \\ A = {A}_{o} [ [ ( 1 + \frac{β {k}_{1}}{{k}_{1} - {k}_{2} - {k}_{3}} ) {e}^{- ( {k}_{1} + {k}_{3} ) t} ] - [ ( \frac{β {k}_{1}}{{k}_{2} - {k}_{1} - {k}_{3}} ) {e}^{- {k}_{2} t} ] ] & Equation 6 \\ A = {A}_{o} [ ( 1 + \frac{β {k}_{1}}{{k}_{2} - {k}_{1}} ) {e}^{- {k}_{1} t} - \frac{{k}_{1} β}{{k}_{2} - {k}_{1}} {e}^{- {k}_{2} t} ] & Equation 7\end{matrix}$$

The single step inactivation model is the most-simple model investigated within these examples (Equation 5). Where A₀ is the initial activity of the enzyme (assumed to be 100%), A is the activity after exposure to elevated temperatures for time t, and k_{d} is the first-order thermal inactivation rate coefficient at the given temperature (T).

The inactivation rate coefficients (kd) for temperatures between 50 and 80° C. are determined by linearly regressing the residual activity versus time on a semi-log plot, with the slope being kd. The resulting kd values are plotted against temperature to obtain a temperature-dependent inactivation coefficient through exponential regression, resulting in an Arrhenius-type equation and plot (Equation 8).

$${k}_{d} ( T ) = P * {e}^{( \frac{- {E}_{a}}{R * T} )} \qquad Equation 8$$

where T is temperature, P is the pm-exponential factor, E_{α} is the inactivation energy and R is the universal gas constant per the Arrhenius equation.

The parameters associated with the series-parallel and series inactivation models (β, k₁, k₂, and k₃) can be estimated using the nonlinear regression code within MATLAB® (Math Works, Natick, Mass.) called ‘fmincon’ to minimize the difference between experimental and predicted data by varying the inactivation coefficients for each inactivation scheme. Constraints can be added that would only evaluate a specific range of numerical values, for example, reversibility of the reactions can be configured not to be permitted (i.e., no negative k₁, k₂, or k₃ values).

### REFERENCES

Anthon, G.; Barrett, D., Kinetic parameters for the thermal inactivation of quality-related enzymes in carrots and potatoes. Journal of Agricultural and Food Chemistry 2002, 50, 4119-4125.

Aymard, C.; Belarbi, A., Kinetics of thermal deactivation of enzymes: a simple three parameters phenomenological model can describe the decay of enzyme activity, irrespectively of the mechanism. Enzyme and Microbial Technology 2000, 27, (8), 612-618.

Balasubramanian, A.; Ponnuraj, K., Purification, crystallization and preliminary X-ray analysis of urease from pigeon pea (Cajanus cajan). Acta Crystallogrophica Section F-Structural Biology Communications 2008, 64, 662-664.

Benge, G., Improving wellbore seal integrity in CO2 injection wells. Energy Procedia 2009,1, (1), 3523-3529.

Borglum, S. J. and Todd, B. J., “An Investigation of Ancient Geological Events and Localized Fracturing on Current Bakken Trends”, SPE 161331, presented at the 2012 SPE Eastern Regional Meeting, Lexington, Kentucky, Oct. 3-5, 2012

Chivvis, R.; Julian, J.; Cary, D., Pressure-activated sealant economically repairs casing leaks on Prudhoe Bay wells. Society of Peteroleum Engineers, 2009 Western Regional Meeting San Jose, Calif., 2009, pp. 1-11.

Crow, W.; Williams, B. D.; Carey, W. J.; Celia, M.; Gasda, S., Wellbore integrity analysis of a natural CO₂ producer. Energy Procedia 2009, 1, (1), 3561-3569.

Cunningham, A.; Gerlach, R.; Phillips, A.; Lauchnor, E.; Rothman, A.; Hiebert, R.; Busch, A.; Lomans, B.; Spangler, L, Assessing potential for biomineralization sealing in fractured shale at the Mont Terri Underground Research Facility, Switzerland. Book chapter in Carbon Dioxide Capture for Storage in Deep Geologic Formations, Gerdes, K., Ed. CPL Press: Amsterdam, 2015 (anticipated); Vol. 4.

Cunningham, A.; Gerlach, R.; Spangler, L.; Mitchell, A.; Parks, S.; Phillips, A., Reducing the risk of well bore leakage of CO₂ using engineered biomineralization barriers. Energy Procedia 2011, 4, 5178-5185.

Cunningham, A.; Lauchnor, E.; Eldring, J.; Esposito, R.; Gerlach, R.; Phillips, A.; Ebigbo, A.; Spangler, L., Abandoned well CO₂ leakage mitigation using biologically induced mineralization: current progress and future directions. Greenhouse Gases Science and Technology 2013, 2, 1-10.

Cunningham, A.; Phillips, A.; Troyer, E.; Lauchnor, E.; Hiebert, R.; Gerlach, R.; Spangler, L., Wellbore leakage mitigation using engineered biomineralization. Energy Procedia 2014, 63, (0), 4612-4619.

Cuthbert, M.; McMillan, L.; Handley-Sidhu, S.; Riley, M. S.; Tobler, D.; Phoenix, V., A field and modeling study of fractured rock permeability reduction using microbially induced calcite precipitation. Environmental Science & Technology 2013, 47, (23), 13637-13643.

Das, N.; Kayastha, A. M.; Srivastava, P. K., Purification and characterization of urease from dehusked pigeonpea (Cajanus cajan L.) seeds. Phytochemistry 2002, 61, (5), 513-521.

De Belie, N.; Verstraete, W., Diatomaceous earth as a protective vehicle for bacteria applied for self-healing concrete. Journal of Industrial Microbiology & Biotechnology 2012, 39, (4), 567-577.

De Muynck, W.; De Belie, N.; Verstraete, W., Microbial carbonate precipitation in construction materials: A review. Ecological Engineering 2010, 36, (2), 118-136.

DeJong, J. T.; Soga, K.; Banwart, S. A.; Whalley, W. R.; Ginn, T. R; Nelson, D. C.; Mortensen, B. M.; Martinez, B. C.; Barkouki, T. Soil engineering in vivo: harnessing natural biogeochemical systems for sustainable, multi-functional engineering solutions. Journal of the Royal Society Interface 2011, 8, (54), 1-15.

DeJong, J. T.; Fritzges, M. B.; Nusslein, K., Microbially induced cementation to control sand response to undrained shear. J. Geotech. Geoenviron Eng. 2006, 132, (11), 1381-1392.

Dupraz, S.; Menez, B.; Gouze, P.; Leprovost, R.; Benezeth, P.; Pokrovsky, O.; Guyot, F., Experimental approach of CO₂ biomineralization in deep saline aquifers. Chemical Geology 2009, 265, 54-62.

Dupraz, S.; Parmentier, M.; Menez, B.; Guyot, F., Experimental and numerical modeling of bacterially induced pH increase and calcite precipitation in saline aquifers. Chemical Geology 2009, 265, 44-53.

Ebigbo, A.; Helmig, R.; Cunningham, A. B.; Class, H.; Gerlach, R., Modelling biofilm growth in the presence of carbon dioxide and water flow in the subsurface. Advances in Water Resources 2010, 33, (7), 762-781.

Ebigbo, A.; Phillips, A.; Gerlach, R.; Helmig, R; Cunningham, A.; Class, H.; Spangler, L., Darcy-scale modeling of microbially induced carbonate mineral precipitation in sand columns. Water Resources Research 2012, 48, (7), W07519.

Ferris, F.; Phoenix, V.; Fujita, Y.; Smith, R., Kinetics ofcalcite precipitation induced by ureolytic bacteria at 10 to 20 degrees C. in artificial groundwater. Geochimica Et Cosmochimica Acta 2003, 67, (8), 1701-1710.

Flemisch, B.; Darcis, M.; Erbertseder, K.; Faigle, B.; Lauser, A.; Mosthaf, K.; Müthing, S.; Nuske, P.; Tatomir, A.; Wolff, M.; Helmig, R., DuMu^{x}: DUNE for multi{phase, component, scale, physics, . . . } flow and transport in porous media. Advances in Water Resources 2011, 34, (9), 1102-1112.

Fujita, Y.; Taylor, J.; Gresham, T.; Delwiche, M.; Colwell, F.; McLing, T.; Petzke, L.; Smith, R., Stimulation of microbial urea hydrolysis in groundwater to enhance calcite precipitation. Environmental Science & Technology 2008, 42, 3025-3032.

Fujita, Y.; Taylor, J.; Wendt, L.; Reed, D.; Smith, R., Evaluating the potential of native ueolytic microbes to remediate a (90)Sr contaminated environment. Environmental Science & Technology 2010, 44, (19), 7652-7658.

Fujita, Y.; Redden, G.; Ingram, J.; Cortez, M.; Ferris, F.; Smith, R., Strontium incorporation into calcite generated by bacterial ureolysis. Geochimica et cosmochimica acta 2004, 68, (15), 3261.

Gasda, S. E.; Bachu, S.; Celia, M. A., Spatial characterization of the location of potentially leaky wells penetrating a deep saline aquifer in a mature sedimentary basin. Environmental Geology 2004, 46, (6/7), 707-720.

Gomez, M. G.; Martinez, B. C.; DeJong, J. T.; Hunt, C. E.; deVlarning, L. A.; Major, D. W.; Dworatzek, S. M., Field-scale bio-cementation tests to improve sands. Proceedings of the Institution of Civil Engineers-Ground Improvement 2015, 168, (3), 206-216.

Grancic, P.; Illeova, V.; Polakovic, M.; Sefcik, J., Thermally induced inactivation and aggregation of urease: Experiments and population balance modelling. Chemical Engineering Science 2012, 70, 14-21.

Hamdan, N.; Kavazanjian, E. J., Enzyme-induced carbonate mineral precipitation for fugitive dust control Geotechnique 2016, 66 (7) 546-555.

Handley-Sidhu, S.; Sham, E.; Cuthbert, M. 0.; Nougarol, S.; Mantle, M.; Johns, M. L.; Macaskie, L. E.; Renshaw, J. C., Kinetics of urease mediated calcite precipitation and permeability reduction of porous media evidenced by magnetic resonance imaging. International Journal of Environmental Science and Technology 2013, 10, (5), 881-890.

Harkes, M.; van Paassen, L.; Booster, J.; Whiffin, V.; van Loosdrecht, M., Fixation and distribution of bacterial activity in sand to induce carbonate precipitation for ground reinforcement. Ecological Engineering 2010, 36, (2), 112-117.

Harris, K.; Johnson, B. Successful remedial operations using ultrafine cement, Mid-Continent Gas Symposium, Amarillo, Tex., 1992; Society of Petroleum Engineers: Amarillo, Tex., 1992; pp 21-30.

Harrison, W.; Luza, K. Temperature gradient information for several boreholes drilled in Oklahoma; Oklahoma Geological Survey: 1986; p 42.

Henley, J. P.; Sadana, A., A mathematical-analysis of enzyme stabilization by a series type mechanism—influence of chemical modifiers. Biotechnol. Bioeng. 1984, 26, (8), 959-969.

Herigstad, B.; Hamilton, M.; Heersink, J. How to optimize the drop plate method for enumerating bacteria. J. Microbiol. Methods 2001, 44 (2),121-129.

Hommel, J.; Cunningham, A. B.; Helmig, R.; Ebigbo, A.; Class, H., Numerical investigation of microbially induced calcite precipitation as a leakage mitigation technology. Energy Procedia 2013, 40, (0), 392-397.

Hommel, J.; Lauchnor, E.; Gerlach, R.; Cunningham, A. B.; Ebigbo, A.; Helmig, R; Class, H. (2015): Investigating the influence of the initial biomass distribution and injection strategies on biofilm-mediated calcite precipitation in porous media. Transport in Porous Media. Pp. 1-23. DOI: 10.1007/s11242-015-0617-3

Hommel, J.; Lauchnor, E.; Phillips, A.; Gerlach, R; Cunningham, A. B.; Helmig, R.; Ebigbo, A.; Class, H., A revised model for microbially induced calcite precipitation—improvements and new insights based on recent experiments. Water Resources Research 2015, DOI: 10.100212014W8016503

Illeová V, Polakovic M, Stefuca V, Acai P, Juma M. Experimental modelling of thermal inactivation of urease. J Biotechnol. 2003, 105(3):235-43.

Jones, P.; Karcher, J.; Ruch, A.; Beamer, A.; Smit, P.; Hines, S.; Olson, M.; Day, D., Rigless operation to restore wellbore integrity using synthetic-based resin sealants. Society of Petroleum Engineers SPE/EAGE European Unconventional Resources Conference and Exhibition, 2014 Vienna, Austria, pg 1-9.

Jung D, Biggs H, Erikson J, Ledyard PU. New Colorimetric reaction for end-point, continuous-flow, and kinetic measurement of urea. Clin Chem. 1975 21(8):1136-40.

Krajewska, B., Ureases I. Functional, catalytic and kinetic properties: A review. Journal of Molecular Catalysis B-Enzymatic 2009, 59, (1-3), 9-21.

Kupresan, D; Heathman, J; Radonjic, M; Application of a new physical model of expandable casing technology in mitigation of wellbore leaks, Journal of Canadian Energy Technology & Innovation 2013, 1(5), 21-24.

Lauchnor E. G., Schultz, L, Bugni, S., Mitchell, A. C., Cunningham, A. B., and Gerlach, R. Bacterially induced calcium carbonate precipitation and strontium co-precipitation in a porous media flow system. Environmental Science and Technology 2013, 47 (3), 1557-1564.

Lauchnor, E.; Topp, D.; Parker, A.; Gerlach, R, Whole cell kinetics of ureolysis by Sporosarcina pasteurii. Journal of Applied Microbiology 2015, 118 (6), 1321-1332.

Lizak, K.; Zeltmann, T.; Crook, R. Permian Basin Operators Seal Casing Leaks with Small-Particle Cement, Permian Basin Oil and Gas Recovery Conference, Midland, Texas, USA, Society of Petroleum Engineers: Midland, Tex., USA, 1992; pp 429-434.

Mateer, J. G.; Marshall, E. K., The urease content ofcertain beans, with special reference to the jack bean. Journal of Biological Chemistry 1916, 25, (2), 297-305.

Mitchell, A. C.; Dideriksen, K.; Spangler, L. H.; Cunningham, A. B.; Gerlach, R., Microbially enhanced carbon capture and storage by mineral-trapping and solubility-trapping. Environ Sci. Technol. 2010, 44, (13), 5270-5276.

Mitchell, A. C.; Ferris, F. G., The influence of Bacillus pasteurii on the nucleation and growth ofcalcium carbonate. Geomicrobiology Journal 2006, 23, (3-4), 213-226.

Mitchell, A C, Phillips, A J, Hiebert, R, Gerlach, R, Cunningham, A, Spangler, L. 2009. Biofilm enhanced geologic sequestration of supercritical CO₂, International Journal of Greenhouse Gas Control, 3: 90-99

Mitchell, A C, Phillips, A J, Kaszuba, J, Hollis, H K, Gerlach, R, Cunningham, A. 2008. Resilience of planktonic and biofilm cultures to supercritical CO2, Journal of Supercritical Fluids, 47: 318-325.

Okwadha, G.; Li, J., Biocontainment of polychlorinated biphenyls (PCBs) on flat concrete surfaces by microbial carbonate precipitation. Journal of Environmental Management 2011, 92, (10), 2860-2864.

Opedal, N. T.; Torsaeter, M.; Vrålstad, T.; Cerasi, P., Potential leakage paths along cement-formation interfaces in wellbores; Implications for CO₂ storage. Energy Procedia 2014, 51, (0), 56-64.

Phillips, A. Biofilm-induced calcium carbonate precipitation: Application in the subsurface. PhD Dissertation, College of Engineering, Montana State University, 2013.

Phillips A J, Cunningham, A, Gerlach, R, Hiebert, R, Hwang, C, Lomans, B, Westrich, J, Mantilla, C, Kirksey, J, Esposito, R, and Spangler, L. 2016. Fracture sealing with microbially-induced calcium carbonate precipitation: A field study. Environmental Science and Technology, 50 (7), pp 4111-4117.

Phillips, A.; Eldring, J.; Hiebert, R.; Lauchnor, E.; Mitchell, A.; Cunningham, A.; Spangler, L.; Gerlach, R., Design of a meso-scale high pressure vessel for the laboratory examination of biogeochemical subsurface processes. Journal of Petroleum Science and Engineering 2015, 126, (0), 55-62.

Phillips, A.; Gerlach, R.; Lauchnor, E.; Mitchell, A.; Cunningham, A.; Spangler, L., Engineered applications of ureolytic biomineralization: a review. Biofouling 2013, 29, (6), 715-733.

Phillips, A. J.; Lauchnor, E.; Eldring, J.; Esposito, R.; Mitchell, A.; Gerlach, R.; Cunningham, A.; Spangler, L., Potential CO₂ leakage reduction through biofilm-induced calcium carbonate precipitation. Environmental Science & Technology 2013, 47, (1), 142-149.

Phillips, A. J.; Troyer, E.; Hiebert, R.; Kirkland, C.; Gerlach, R.; Cunningham, A. B.; Spangler, L.; Kirksey, J.; Rowe, W.; Esposito, R., Enhancing wellbore cement integrity with microbially induced calcite precipitation (MICP): A field scale demonstration. Journal of Petroleum Science and Engineering 2018, 171, 1141-1148.

Polacco, J. C.; Havir, E. A., Comparisons of soybean urease isolated from seed and tissue culture. Journal of Biological Chemistry 1979, 254, (5), 1707-1715.

Polakovic, M.; Vrabel, P., Analysis of the mechanism and kinetics of thermal inactivation of enzymes: Critical assessment of isothermal inactivation experiments. Process Biochemistry 1996, 31, (8), 787-800.

Polakovic, M.; Vrabel, P.; Bales, V., Approaches for improved identification of mechanisms of enzyme inactivation. In Stability and Stabilization of Biocatalysts, Ballesteros, A.; Plou, F. J.; Iborra, J. L.; Hailing, P. J., Eds. Elsevier Science By: Amsterdam, 1998; Vol. 15, pp 77-82.

Martinez, B.; DeJong, J.; Ginn, T.; Montoya, B.; Barkouki, T.; Hunt, C.; Tanyu, B.; Major, D., Experimental optimization of microbial-induced carbonate precipitation for soil improvement. Journal of Geotechnical and Geoenvironmental Engineering 2013, 139, (4), 587-598.

Menegassi, A.; Wassermann, G. E.; Olivera-Severo, D.; Becker-Ritt, A. B.; Martinelli, A. H. S.; Feder, V.; Carlini, C. R, Urease from cotton (Gossypium hirsutum) seeds: Isolation, physicochemical characterization, and antifungal properties of the protein. Journal of Agricultural and Food Chemistry 2008, 56, (12), 4399-4405.

Mitchell, A. C.; Ferris, F. G., The influence of Bacillus pasteurii on the nucleation and growth of calcium carbonate. Geomicrobiology Journal 2006, 23, (3-4), 213-226.

Mitchell, A.; Phillips, A.; Kaszuba, J.; Hollis, W.; Cunningham, A.; Gerlach, R., Microbially enhanced carbonate mineralization and the geologic containment of CO₂. Geochimica Et Cosmochimica Acta 2008, 72, (12), A636-A636.

Mitchell, A.; Phillips, A.; Schultz, L.; Parks, S.; Spangler, L.; Cunningham, A.; Gerlach, R., Microbial CaCO₃ mineral formation and stability in an experimentally simulated high pressure saline aquifer with supercritical CO₂. International Journal of Greenhouse Gas Control 2013,15, 86-96.

Norris, R.; Brocklehurst, K., Convenient method of preparation of high-activity urease from Canavalia ensiformis by covalent chromatography and an investigation of its thiol-groups with 2,2′-dipyridyl disulfide as a thiol titratnt and reactivity probe. Biochemical Journal 1976, 159, (2), 245-257.

Real-Guerra, R.; Staniscuaski, F.; Carlini, C. R., Soybean urease: Over a hundred years of knowledge. Comprehensive Survey of International Soybean Research—Genetics, Physiology, Agronomy and Nitrogen Relationships 2013, 317-339.

Sadana, A., Enzyme deactivation. Biotechnology Advances 1988, 6, (3), 349-446.

Scorsone, J.; Sanders, M.; Patel, A., An improved oil-based chemical gel system for wellbore stabilization. Society of Petroleum Engineers SPE International Symposium on Oilfield Chemistry, 20-22 April, The Woodlands, Texas, 2009, pp. 1-5.

Sizer, I. W., Activation energy of urea hydrolysis catalyzed by soy bean urease. Journal of Biological Chemistry 1940, 132, (1), 209-218.

Stocks-Fischer, S.; Galinat, J. K.; Bang, S. S., Microbiological precipitation of CaCO₃. Soil Biology & Biochemistry 1999, 31, (11), 1563-1571.

Sujoy, B.; Apama, A., Enzymology, immobilization and applications of urease enzyme. International Research Journal of Biological Sciences 2013, 2, (6), 51-56.

Sumner, J. B., The isolation and crystallization of the enzyme urease. Preliminary paper. Journal of Biological Chemistry 1926, 69, (2), 435-441.

Tahany, M. A.; Abdel-Rahman, M.; Salama, A.-A.; Aly, M. I. A.; El-Selhdar, A. A. H., Preparation and kinetics of urease in A. fumigatus and F. nivale. Egyt. Journal of Microbiology 1992.

van Paassen, L.; Daza, C.; Staal, M.; Sorokin, D. Y.; van der Zon, W.; van Loosdrecht, M., Potential soil reinforcement by biological denitrification. Ecological Engineering 2010, 36, (2), 168-175.

van Paassen, L.; Ghose, R.; van der Linden, T.; van der Star, W.; van Loosdrecht, M., Quantifying biomediated ground improvement by ureolysis: large-scale biogrout experiment. Journal of Geotechnical and Geoenvironmental Engineering 2010, 136, (12), 1721-1728.

Van Tittelboom, K.; De Belie, N.; De Muynck, W.; Verstraete, W., Use of bacteria to repair cracks in concrete. Cement and Concrete Research 2010, 40, (1), 157-166.

Tobler, D. J.; Maclachlan, E.; Phoenix, V. R., Microbially mediated plugging of porous media and the impact of differing injection strategies. Ecol. Eng. 2012, 42, 270-278.

Tobler, D. J.; Cuthbert, M. O.; Greswell, R. B.; Riley, M. S.; Renshaw, J. C.; Handley Sidhu, S.; Phoenix, V. R., Comparison of rates of ureolysis between Sporosarcina pasteurii and an indigenous groundwater community under conditions required to precipitate large volumes of calcite. Geochimica et Cosmochimica Acta 2011, 75, (11), 3290-3301.

Watson, T. L.; Bachu, S., Evaluation of the potential for gas and CO₂ leakage along wellbores. SPE Drilling and Completion 2009, 24, (01), 1115-126.

Whiffin, V.; van Paassen, L.; Harkes, M., Microbial carbonate precipitation as a soil improvement technique. Geomicrobiology Journal 2007, 24, 417-423.

Wigand, M.; Kaszuba, J. P.; Carey, J. W.; Hollis, W. K., Geochemical effects of CO₂ sequestration on fractured wellbore cement at the cement/caprock interface. Chemical Geology 2009, 265, (1-2), 122-133.

Wollenweber, J.; Alles, S.; Busch, A.; Krooss, B.; Stanjek, H.; Littke, R., Experimental investigation of the CO₂ sealing efficiency of caprocks. International Journal of Greenhouse Gas Control 2010, 4, (2), 231-241.

1. A method of precipitating calcium carbonate at a location, the method comprising introducing urea, calcium, and a heat-treated cell preparation comprising active urease enzyme to the location, wherein the urease enzyme hydrolyzes the urea to ammonium carbonate, and the calcium reacts with the carbonate to form a calcium carbonate precipitate at the location.

2. The method of claim 1, wherein the heat-treated cell preparation includes, in addition to the active urease enzyme, heat-treated cells in the form of intact cells, non-intact remnants thereof, or a mixture thereof.

3. The method of claim 2, wherein the heat-treated cell preparation is produced by heating a urease-producing cell preparation.

4. The method of claim 3, wherein the heating comprises heating the urease-producing cell preparation at a temperature from about 50° C. to about 70° C. for a time from about 1 minute to about 30 minutes.

5. The method of claim 3, wherein the urease-producing cell preparation comprises a urease-producing microbe.

6. The method of claim 5, wherein the urease-producing microbe comprises Sporosarcina pasteurii.

7. The method of claim 3, wherein the heating is sufficient to inactivate at least a portion of the cells in the preparation while maintaining at least some of the activity of the urease made by the cells.

8. The method of claim 3, wherein the heating inactivates at least about 95% of the urease-producing cells in the urease-producing cell preparation.

9. The method of claim 3, wherein the heating increases urease activity in the heat-treated cell preparation with respect to urease activity in the urease-producing cell preparation.

10. The method of claim 3, wherein the heat-treated cell preparation is introduced to the location after the heating without purification or isolation of components therefrom.

11. The method of claim 3, wherein at least one of the urea and the calcium is introduced to the location in a fluid preparation separate from the heat-treated cell preparation.

12. The method of claim 3, wherein the location comprises a channel, and the urea, the calcium, and the heat-treated cell preparation is introduced into the channel in amounts and for a time sufficient to at least partially seal the channel.

13. The method of claim 12, wherein the channel is a subterranean channel.

14. The method of claim 12, wherein the channel is in fluid communication with a wellbore.

15. The method of claim 12, wherein the channel comprises a channel in a subterranean formation.

16. The method of claim 12, wherein the channel comprises a channel between a cement sheath and a well casing of a subterranean wellbore.

17. The method of claim 12, wherein the channel comprises a channel between a cement sheath surrounding a well casing of a subterranean wellbore and a subterranean formation through which the subterranean wellbore is drilled.

18. The method of claim 12, wherein the channel comprises a crack in a cement sheath surrounding a well casing of a subterranean wellbore.

19. A method of preparing a heat-treated cell preparation, the method comprising heating a urease-producing cell preparation at a temperature and for a time sufficient to inactivate at least a portion of the cells in the urease-producing cell preparation while maintaining at least some urease activity of urease made by the cells in the urease-producing cell preparation.

20. The method of claim 19, wherein the heating comprises heating the urease-producing cell preparation at a temperature from about 50° C. to about 70° C. for a time from about 1 minute to about 30 minutes.

21. The method of claim 19, wherein the urease-producing cell preparation comprises a urease-producing microbe.

22. The method of claim 21, wherein the urease-producing microbe comprises Sporosarcina pasteurii.

23. The method of claim 19, wherein the heating inactivates at least about 95% of the urease-producing cells in the urease-producing cell preparation.

24. The method of claim 19, wherein the heating increases urease activity in the heat-treated cell preparation with respect to urease activity in the urease-producing cell preparation.

25. The method of claim 19, further comprising, after the heating, cooling the heat-treated cell preparation to a temperature below about 40° C.
